(19) United States
(12) Patent Application Publication
(10) Pub. No.: US 2024/0016968 A1
(43) Pub. Date: Jan. 18, 2024

(54) OZONE PURIFICATION DEVICE

(71) Applicant: Microjet Technology Co., Ltd., Hsinchu (TW)

(72) Inventors: Hao-Jan Mou, Hsinchu (TW); Chin-Chuan Wu, Hsinchu (TW); Yung-Lung Han, Hsinchu (TW); Chi-Feng Huang, Hsinchu (TW)

(73) Assignee: Microjet Technology Co., Ltd., Hsinchu (TW)

(21) Appl. No.: 18/134,177

(22) Filed: Apr. 13, 2023

(30) Foreign Application Priority Data
Jul. 15, 2022 (TW) 111126753

(51) Int. Cl. A61L 9/20

(52) U.S. Cl. CPC A61L 9/205; A61L 2202/11; A61L 2202/13; A61L 2209/12; A61L 2209/212

(57) ABSTRACT

An ozone purification device is disclosed. The ozone purification device comprises an ultraviolet light source and a filter. The ultraviolet light source includes an ultraviolet lamp and a LED lamp. The ultraviolet lamp generates ultraviolet rays and the LED lamp generates heat. The filter is composed of a metal composite catalyst coated with an active carbon. When ozone passes through the ultraviolet light source, ozone is irradiated by the ultraviolet light lamp and the LED lamp of the ultraviolet light source, and then passes through the metal composite catalyst and the active carbon of the filter to accelerate the decomposition of ozone into oxygen.

### CROSS-REFERENCE TO RELATED APPLICATION

This application claims priority to Taiwan Patent Application No. 111126753, filed on Jul. 15, 2022. The entire contents of the above-mentioned patent application are incorporated herein by reference for all purposes.

### FIELD OF THE INVENTION

The present disclosure relates to a purification device, and more particularly to an ozone purification device for purifying ozone.

### BACKGROUND OF THE INVENTION

In modern society, people are increasingly concerned about the quality of gases in their living environment.

Although ozone can utilize its strong oxidation property to reach the effect of sterilization and deodorization, when its content in the air is too high, exposure to the environment may have a significant impact on human health. For example, it may irritate the respiratory tract, cause asthma, and in severe cases, it may damage human respiratory system and cause emphysema, pulmonary edema and other problems, and even affect the efficiency of blood oxygen supply. In Taiwan, the air quality standard is to limit the ozone concentration to less than 0.12 ppm per hour (volume concentration per million), and the eight-hour average should be less than 0.06 ppm. Therefore, how to eliminate excessive ozone in an airtight indoor space has been the main subject of the present disclosure.

### SUMMARY OF THE INVENTION

An object of the present disclosure is to provide an ozone purification device. In accordance with an aspect of the present disclosure, the ozone purification device comprises an ultraviolet light source and a filter. The ultraviolet light source includes an ultraviolet lamp and a LED lamp. The ultraviolet lamp generates ultraviolet rays and the LED lamp generates heat. The filter is composed of a metal composite catalyst coated with an active carbon. When ozone passes through the ultraviolet light source, ozone is irradiated by the ultraviolet lamp and the LED lamp of the ultraviolet light source, and then passes through the metal composite catalyst and the active carbon of the filter to accelerate the decomposition of ozone into oxygen.

### BRIEF DESCRIPTION OF THE DRAWINGS

The above contents of the present disclosure will become more readily apparent to those ordinarily skilled in the art after reviewing the following detailed description and accompanying drawings, in which:

FIG. 1 is a schematic perspective view illustrating an ozone purification device according to an embodiment of the present invention.

### DETAILED DESCRIPTION OF THE PREFERRED EMBODIMENT

The present disclosure will now be described more specifically with reference to the following embodiments. It is to be noted that the following descriptions of preferred embodiments of this invention are presented herein for purpose of illustration and description only. It is not intended to be exhaustive or to be limited to the precise form disclosed.

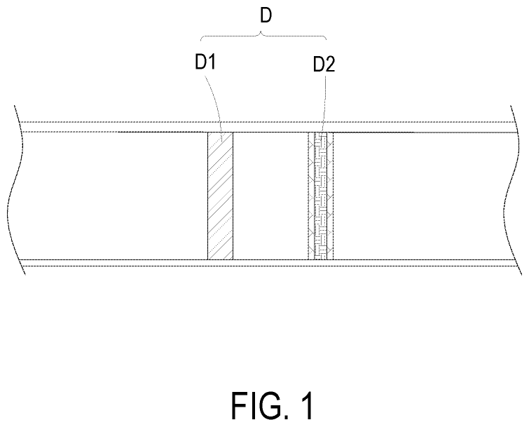

Please refer to FIG. 1. The present disclosure provides an ozone purification device D, which includes an ultraviolet light source D1 and a filter D2. The ultraviolet light source D1 includes an ultraviolet lamp and a LED lamp. The ultraviolet lamp generates ultraviolet rays, and the LED lamp generates heat. The filter D2 is composed of a metal composite catalyst coated with an active carbon. When ozone passes through the ultraviolet light source D1, ozone is irradiated by the ultraviolet lamp and the LED lamp of the ultraviolet light source D1. Then, the ozone passes through the metal composite catalyst and the active carbon of the filter D2 to accelerate the decomposition of ozone into oxygen, so as to achieve the ozone purification function.

Notably, the luminous wavelength of the ultraviolet lamp is from the band of short wavelength 100 nm to 280 nm. In the present disclosure, the best embodiment of the luminous wavelength of the ultraviolet lamp of ultraviolet light source D1 is 265 nm, but not limited thereto, and the luminous wavelength can be adjustable according the practical requirement. In addition, it is to be noted that the LED lamp is mainly used as a heat source, but not limited thereto, which can also be replaced with a heat source element, such as a resistor or a high-impedance element.

Moreover, the irradiation function of the ultraviolet light source D1 is to provide 30% of energy of the ultraviolet lamp to generate ultraviolet rays, and provide 70% of energy of the LED lamp to generate heat, but not limited thereto, the energy distribution ratio of the ultraviolet lamp and the LED lamp can be adjustable according the practical requirement.

In a first embodiment of the present disclosure, the metal composite catalyst of the filter D2 is composed of a manganese oxide and at least one transition metal oxide. Notably, the transition metal can be a known transition metal in the technical field, which includes titanium (Ti), chromium (Cr), iron (Fe), cobalt (Co), nickel (Ni), copper (Cu), zirconium (Zr), niobium (Nb), molybdenum (Mo), vanadium (V), scandium (Sc), or a mixture of at least two of them, but not limited thereto, which can be adjustable according the practical requirement.

In a second embodiment of the present disclosure, the metal composite catalyst of the filter D2 is composed of a manganese oxide and a noble metal palladium (P d). Notably, the noble metal can be a known noble metal in the technical field, which includes gold (Au), silver (Ag), platinum (Pt), palladium (Pd), but not limited thereto, which can be adjustable according the practical requirement.

In a third embodiment of the present disclosure, the metal composite catalyst of the filter D2 is composed of a manganese(Mn) oxide and a cerium (C e).

In a fourth embodiment of the present disclosure, the metal composite catalyst of the filter D2 is composed of an iron-modified zeolite and an iron-modified quartz sand. Notably, the iron-modified zeolite and the iron-modified quartz sand can be mixed in a mass ratio of 1:4 to 4:1, but not limited thereto, the mass ratio of the iron-modified zeolite and the iron-modified quartz sand can be adjustable according the practical requirement.

In any embodiment of the present disclosure, an activated carbon coated on the surface of the metal composite catalyst is combined with the metal composite catalyst to form the filter D2. Notably, the ozone is irradiated by the ultraviolet light generated by the ultraviolet lamp and the heat generated by the LED lamp, and passes through the metal composite catalyst on the surface of the filter D2. Since the half-life of ozone in the air is about 20-30 minutes, ozone is irradiated by the ultraviolet rays generated by 30% of the energy provided by the ultraviolet lamp, and photolysis and sterilization are carried out through the ultraviolet rays. At the same time, ozone is also irradiated by the heat generated by 70% of the energy provided by the LED lamp, which increases the temperature and accelerates the photolysis speed. When the temperature reaches about 60° C., the ozone will be quickly reduced to oxygen, and the ozone can also be accelerated to be reduced to oxygen by contacting the metal composite catalyst on the surface of the filter D2. Consequently, the ozone can accelerate the process of reducing to oxygen through the three-in-one decomposition of the ultraviolet sterilization, the LED lamp heating and the metal composite catalyst. Moreover, the activated carbon is mainly to adsorb inorganic or organic compounds contained in the gas, to adsorb toxic components in the gas or components that are harmful to the human body, or to remove odorous components, so that the bacteria or viruses killed by ozone or entrained particles of ozone can be filtered by the activated carbon.

In summary, the present disclosure provides an ozone purification device. The ozone purification device is used for purifying ozone by the ultraviolet sterilization produced by the ultraviolet lamp, the heat generated by the LED lamp, and the metal composite catalyst, so as to reach the effect of the three-in-one decomposition, and to accelerate the cleaning and purifying effect of reducing to oxygen.

What is claimed is:

1. An ozone purification device comprising:
an ultraviolet light source including an ultraviolet lamp and a LED lamp, wherein the ultraviolet lamp generates ultraviolet rays and the LED lamp generates heat; and
a filter being composed of a metal composite catalyst coated with an active carbon,
wherein when ozone passes through the ultraviolet light source, ozone is irradiated by the ultraviolet lamp and the LED lamp of the ultraviolet light source, and then passes through the metal composite catalyst and the active carbon of the filter to accelerate the decomposition of ozone into oxygen.

2. The ozone purification device according to claim 1, wherein the irradiation function of the ultraviolet light source is to provide 30% of an energy of the ultraviolet lamp to generate ultraviolet rays, and provide 70% of an energy of the LED lamp to generate heat.

3. The ozone purification device according to claim 1, wherein a wavelength of ultraviolet rays emitted by the ultraviolet lamp is 265 nm.

4. The ozone purification device according to claim 1, wherein the metal composite catalyst of the filter is composed of a manganese oxide and at least one transition metal oxide.

5. The ozone purification device according to claim 1, wherein the metal composite catalyst of the filter is composed of a manganese oxide and a noble metal palladium.

6. The ozone purification device according to claim 1, wherein the metal composite catalyst of the filter is composed of a manganese oxide and a cerium.

7. The ozone purification device according to claim 1, wherein the metal composite catalyst of the filter is composed of an iron-modified zeolite and an iron-modified quartz sand.
